(19) United States
(12) Patent Application Publication
(10) Pub. No.: US 2016/0089076 A1
(43) Pub. Date: Mar. 31, 2016

(54) WEARABLE DEVICE HAVING ACTIVE FEEDBACK CHARACTERISTICS FOR PHYSIOLOGICAL NURSING, AND SYSTEM AND METHOD THEREOF

(71) Applicant: FLUIDITECH IP LIMITED, Victoria, SC (US)

(72) Inventor: JONATHAN TSAI, Seattle, WA (US)

(21) Appl. No.: 14/616,066

(22) Filed: Feb. 6, 2015

(30) Foreign Application Priority Data
Sep. 25, 2014 (TW) 103133376

(51) Int. Cl. A61B 5/00; A61B 5/11

(52) U.S. Cl. CPC A61B 5/486; A61B 5/681; A61B 5/7405; A61B 5/7455; A61B 5/4839; A61B 5/002; A61B 5/0022; A61B 5/1112; A61B 5/746

(57) ABSTRACT

A wearable device having active feedback characteristics for physiological nursing, and a system and a method thereof are provided. A physiological sensing device is configured to sense a physiological sensing signal of a wearer, and the wearable device actively applies a preset functional response corresponding to the wearer when the physiological sensing signal is not within a safe range to enable the physiological sensing signal returning to a stable state. Disclosed actively and determinedly provides an instant response which enables the wearer's physiological condition being controlled in a relative stable state, providing a pretreatment in no time to wait the follow-up aid, or deciding to do other treatments independently.

### CROSS-REFERENCE TO RELATED APPLICATION

This application claims priority from Taiwan Patent Application No. 103133376, filed on Sep. 25, 2014, in the Taiwan Intellectual Property Office, the content of which are hereby incorporated by reference in their entirety for all purposes.

### BACKGROUND OF THE INVENTION

1. Field of the Invention

This application relates to a physiological nursing device, and a system and a method thereof, and more particularly, to a wearable device having active feedback characteristics for physiological nursing, a system and a method thereof.

2. Description of the Related Art

As the trend in the burgeoning technology of network and the miniaturized electronic components, the technique of the Internet of Things (IoT) along with the wearable technology can be applied to human body easier so as to monitor the vital signs in real-time. In addition, as the technology of cloud and network has been popularized, the wearable devices oriented to physiological nursing become more and more popular in human life.

The conventional wearable devices, however, sense one's vital signs through the collected physiological sensing signals, and once the wearer is under certain special conditions such as the unusual raise of blood glucose, sleep apnea, sudden infant death syndrome (SIDS), and so on, the wearer is unable to ask for help as the unconsciousness. Although the conventional wearable devices are able to actively send e-mails, messages or a call for aid, it is well-known that one may likely be in coma in the abnormal physiological conditions such as in dyspnea about 3-5 minutes. If the period of dyspnea lasts out, the life may be gone. Moreover, if the unusual raise of blood glucose occurs to the wearer, syndrome such as retinopathy, arteriosclerosis, nephropathy, and so on may become inevitable.

In conclusion, the lack of instant physiological nursing is indeed one of the drawbacks of the prior technology. No matter how accurate the sensed result is, the regret is unavoidable if an immediate physiological nursing cannot be provided adequately to the human body.

### SUMMARY OF THE INVENTION

One objective of the present invention is to provide an active and instant physiological nursing.

The other objective of the present invention is to greatly reduce the damage to human's physiological condition when one wears a wearable device having active feedback characteristics for physiological nursing in accordance with the present invention.

For the sake of accomplishing the preceding objectives, the present invention is to provide a wearable device having active feedback characteristics for physiological nursing which may include a physiological signal sensing device being wearable to a wearer, and a control device connected with the physiological signal sensing device; the wearable device having active feedback characteristics for physiological nursing further comprising a feedback device being wearable to the wearer, and the feedback device connected with the control device; when the control device determines a physiological sensing signal produced by the physiological signal sensing device that is not within a safe range, the control device configured to command the feedback device applying a preset functional response to the wearer, wherein the preset functional response may enable the physiological sensing signal of the wearer returning to a stable state so as to be maintained within the safe range.

According to an embodiment in accordance with the present invention, the feedback device may include at least one of a sound player, a vibrator, a current releaser, and a medicine releaser, wherein a preset functional response the sound player may be to play a preset sound, a preset functional response of the vibrator may be to produce a vibration, a preset functional response of the current releaser may be to produce an effect of electric shock, and a preset functional response of the medicine releaser may be to apply a medicine to the wearer.

According to an embodiment in accordance with the present invention, the current releaser may include an anode patch and a cathode patch adhered to the wearer, and the current releaser may release a low frequency current through the anode patch and the cathode patch.

According to an embodiment in accordance with the present invention, a wireless communication module connected with the control device may be further included. Wherein the wireless communication module may be configured to connect with a first electronic communication device outside the wearable device having active feedback characteristics for physiological nursing, such that the physiological signal may be transmitted to the first electronic communication device. Wherein, the wireless communication module may be a near field communication module or a far field communication module.

In order to achieve to the aforementioned objective, the present invention further provides a physiological nursing system applying the above-mentioned wearable device having active feedback characteristics for physiological nursing which may include the wearable device having active feedback characteristics for physiological nursing being wearable to the wearer configured to sense the physiological sensing signal of the wearer and actively commanding the feedback device to produce the preset functional responses to the wearer when the physiological sensing signal is not within the safe range, and the first electronic communication device connected with the wearable device to receive the physiological sensing signal and transmit the physiological sensing signal to a second electronic communication device located in a remote end.

According to an embodiment in accordance with the present invention, the first electronic communication device may be configured with a global positioning system and a mobile communication module, and when the physiological sensing signal is not within the safe range, the first electronic communication device may transmit the physiological sensing signal and position data produced by the global positioning system to the second electronic communication device through the mobile communication module.

In order to achieve to the aforementioned objective as well as the other objectives, the present invention further provides a method having active feedback characteristics for physiological nursing which may include sensing a physiological condition of a wearer to produce a physiological sensing signal, determining whether the physiological sensing signal is not within a safe range, and actively applying a preset functional response relative to the physiological condition of the wearer through a feedback device when the physiological sensing signal is not within the safe range, wherein the preset functional response may enable the physiological sensing signal of the wearer returning to a stable state.

According to an embodiment in accordance with the present invention, the position data may be produced through a global positioning system, and the physiological sensing signal may be simultaneously transmitted to a remote end as an alert when the physiological sensing signal is not within the safe range, wherein the remote end may be at least one of an electric communication device of a preset contactor and a nearby medical institute provided by the position data.

According to an embodiment in accordance with the present invention, in addition to actively applying a preset functional response relative to the physiological condition of the wearer when the physiological sensing signal is not within the safe range, the method may further include regularly transmitting the physiological sensing signal to the remote end, wherein the remote end may be a monitoring host stored with preset physiological data, and the monitoring host may determine whether the preset functional responses are applied to the wearer corresponding to the preset physiological data and the physiological sensing signal when the physiological sensing signal is within the safe range; and if the result is positive, the preset functional response relative to the wearer may be forced to be actively applied through the feedback device.

As a result, if one who wears a wearable device having active feedback characteristics for physiological nursing of the present invention is having physiological problems, the preset functional responses of the arrangement in accordance with the present invention enable the adequate treatment to be actively and immediately provided without waiting passively. For example, if one is in dyspnea and the blood oxygen saturation is much lower than the standard value, closing to be in a shock, the wearable device disclosed in the present invention is able to produce an effect of electric shock by the preset functional response of the current releaser, such that the wearer may recover from the unconsciousness. As to one having abnormality of blood glucose, the wearable device disclosed in the present invention is able to apply the insulin to the wearer by the preset functional response of the medicine releaser, wherein the intake can be injection or the other methods which can be absorbed by human body. The wearable device disclosed in the present invention provides convenient and instant treatment when the wearer is having physiological problems, and the physiological condition of the wearer can therefore be controlled in a relative stable state; and additionally, a wearable device having active feedback characteristics in accordance with the present invention can further provide a pretreatment in no time such that the wearer can wait the follow-up aid, or decide to do other treatments independently.

### BRIEF DESCRIPTION OF THE DRAWINGS

Hereinafter, embodiments of the present invention will be described in detail with reference to the accompanying drawings so that those skilled in the art to which the present invention pertains can realize the present invention, wherein:

FIG. 1 is a block diagram for showing a function of a wearable device having active feedback characteristics in accordance with one embodiment of the present invention.

FIG. 2 is a block diagram for showing a function of a wearable device having active feedback characteristics in accordance with another embodiment of the present invention.

FIG. 3 is a three-dimensional schematic diagram for showing a function of a wearable device having active feedback characteristics in accordance with another embodiment of the present invention.

FIG. 4 is a flow chart of a physiological nursing method having active feedback characteristics in accordance with one embodiment of the present invention.

FIG. 5 is a flow chart of a physiological nursing method having active feedback characteristics in accordance with another embodiment of the present invention.

FIG. 6 is a flow chart of a physiological nursing method having active feedback characteristics in accordance with another embodiment of the present invention.

### DETAILED DESCRIPTION OF THE PREFERRED EMBODIMENTS

Hereinafter, embodiments of the present invention will be described in detail with reference to the accompanying drawings so that those skilled in the art to which the present invention pertains can realize the present invention. As those skilled in the art would realize, the described embodiments may be modified in various different ways, all without departing from the spirit or scope of the present invention.

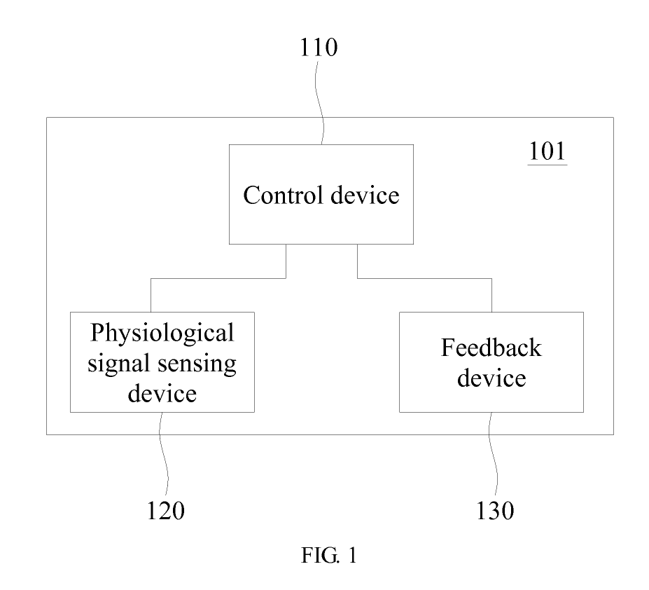

Please refer to FIG. 1 which is a block diagram for showing a function of a wearable device having active feedback characteristics in accordance with one embodiment of the present invention. A wearable device 101 having a physiological signal sensing device 120 of the present invention is feasible to be worn on a wearer, and a control device 110 is connected with the physiological signal sensing device 120. In addition, a feedback device 130 which is wearable is further provided.

The feedback device 130 is connected with the control device 110. The control device 110 is configured to determine whether a physiological sensing signal produced by the physiological signal sensing device 120 is within a safe range. When the control device 110 determines the physiological sensing signal produced by the physiological signal sensing device that is not within a safe range, the control device 110 is configured to actively command the feedback device 130 applying a preset functional response to the wearer. Wherein, the preset functional response enables the physiological sensing signal of the wearer returning to a stable state so as to be maintained within the safe range.

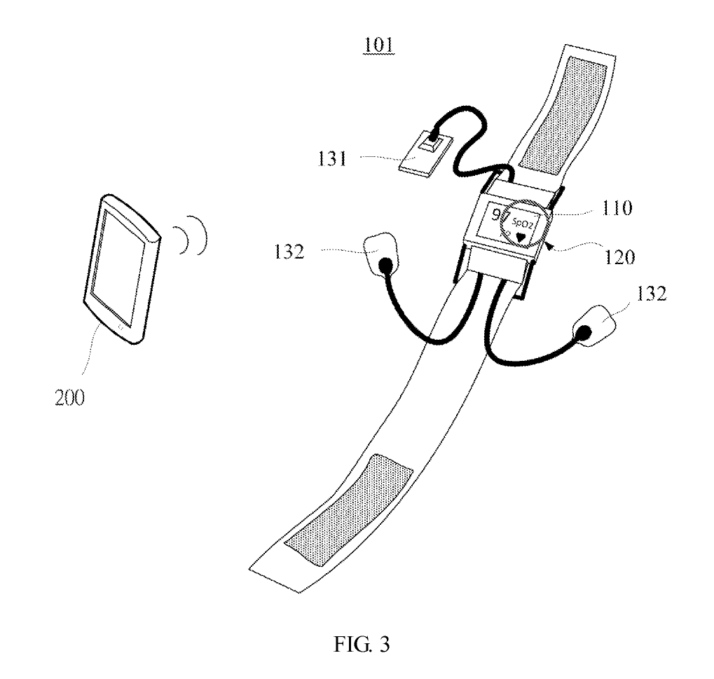

What one skilled in the art can understand is that the control device 110, the physiological signal sensing device 120 and the feedback device 130 may be integrated as a single device in the appearance, or the control device 110 and the physiological signal sensing device 120 may be integrated as a single device (shown in FIG. 3) or the other forms of arrangement. As only one control end, which is mainly configured to control the signals, is necessary to the physiological signal sensing device 120 and the feedback device 130, devices having the related functions do not depart from the scope of the present invention.

The feedback device 130 and the physiological signal sensing device 120 disclosed in the present invention may be able to cooperate with each other, that is to say, when the physiological signal sensing device 120 senses that the physiological condition of the wearer is in danger, the feedback device 130 is configured to apply adequate treatment to the wearer to enable the physiological sensing signal of the wearer returning to a stable state so as to be maintained within the safe range. For example, when the physiological signal sensing device 120 is configured to sense the wearer's blood oxygen saturation (e.g. using infrared to irradiate the wearer's skin, and extracting the reflected light to analyze, or the other sensing devices feasible to obtain the blood oxygen saturation), the setting of the control device 110 relative to the safe range means the normal numeral of blood oxygen saturation. If the numeral of the physiological sensing signal sensed by the physiological signal sensing device 120 is not within the safe range (the numeral is usually set to higher than a threshold value), the control device 110 actively commands the feedback device 130 to release current (e.g. current releaser configured to release a low frequency current through an anode patch and a cathode patch adhered to the wearer) to awaken the wearer's consciousness through the current. As the reduction of blood oxygen saturation is usually caused by dyspnea or insufficient respiratory capacity, the wearer may fall into a shock within 3-5 minutes and the brain organization may hence be damaged if an immediate treatment cannot be done. As a result, the present invention is to apply a physiological nursing system cooperating with the feedback device 130 having active feedback characteristics to provide an adequate treatment instantly according to different physiological conditions so as to seize the precious time for treatment.

The feedback device 130 mentioned in the present invention may include at least one of a sound player, a vibrator, a current releaser, and a medicine releaser. Wherein a preset functional response the sound player is to play a preset sound, a preset functional response of the vibrator is to produce a vibration, a preset functional response of the current releaser is to produce an effect of electric shock caused by an anode patch and a cathode patch, and a preset functional response of the medicine releaser is to apply a medicine to the wearer. Wherein, the medicine releaser may sense the physiological signal relative to the wearer's blood glucose, and when the amount of blood glucose is abnormal, an adequate treatment can be done immediately. For example, the control device 100 may command a medicinal patch to release an adequate amount of insulin which can be absorbed by the wearer's skin so as provide the instant aid. Besides the aforementioned method, injection or the other methods which can be absorbed by human body may be applied to the feedback device 130 as well.

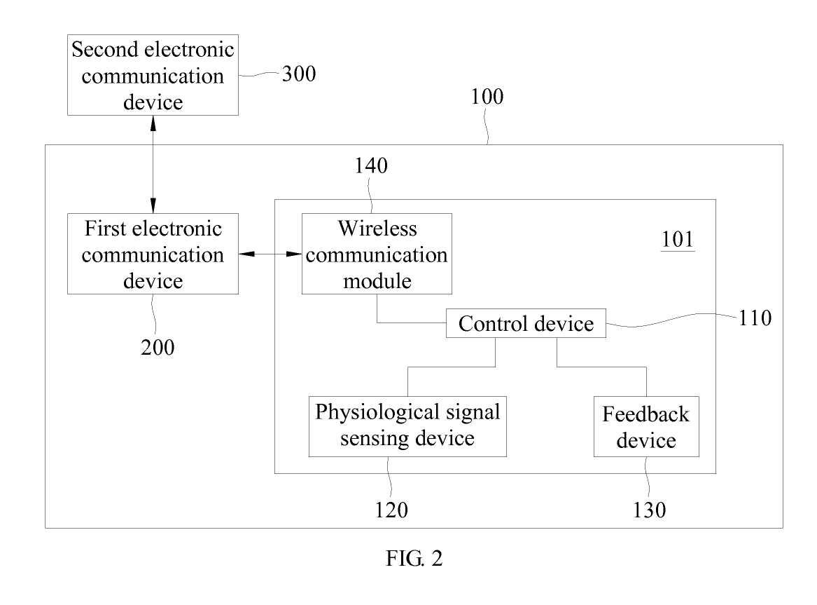

Please refer to FIG. 2 which is a block diagram for showing a function of a wearable device having active feedback characteristics in accordance with another embodiment of the present invention. The embodiment further includes a wireless communication module 140 which is connected with the control device 110 compared with the embodiment of FIG. 1. The wireless communication module 140 is connected with a first electronic communication device 200 such that the physiological signal can be transmitted to the first electronic communication device 200. Wherein, the wireless communication module 140 may be a near field communication module such as NFC, or a far field communication module such as Bluetooth and Wi-Fi connected with the first electronic communication device 200 via wireless connection. On the other hand, the first electronic communication device 200 may be directly connected with the wearable device 101 (not shown) through an universal serial bus (USB) to receive the physiological sensing signal; and additionally, the physiological sensing signal may be transmitted to a second electronic communication device 300 located in a remote end as well. Furthermore, firmware or operational software of the wearable device 101 may be updated through the first electronic communication device 200.

Please refer to FIG. 3 which is a three-dimensional schematic diagram for showing a function of a wearable device having active feedback characteristics in accordance with another embodiment of the present invention. The wearable device 101 may be fixed on the wearer's wrist by tape or Velcro fasteners, and then the physiological signal sensing device 120 may be adjusted in a right sensing part to sense the physiological signal of the wearer. FIG. 3 shows a wearable device having two feedback devices 131, 132. The first feedback device 131 uses a type as an example, and the first feedback device 131 is controlled by the control device 110 to apply the above-mentioned insulin at an appropriate time. The second feedback device 132 takes the two electrode patches used to release a low frequency current as an example; likewise, the second feedback device 132 is controlled by the control device 110 to release current to produce an effect of electric shock at an appropriate time.

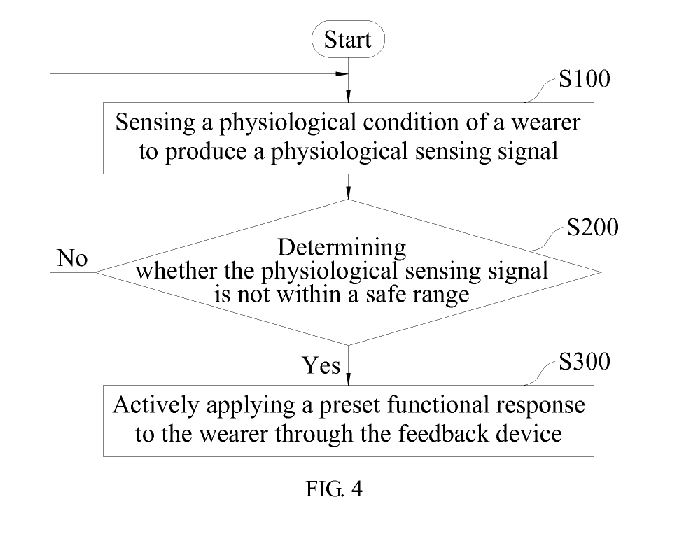

Please refer to FIG. 4 which is a flow chart of a physiological nursing method having active feedback characteristics in accordance with one embodiment of the present invention. As shown in the figure, the operational steps are sated as follow.

In step S100, a physiological condition of a wearer is sensed to produce a physiological sensing signal.

In step S200, whether the physiological sensing signal is determined within a safe range. If the result is “positive”, step S300 is entered, and if the result is “negative”, the procedure backs to step S100.

In step S300, a preset functional response relative to the wearer is actively applied to the wearer through the feedback device when the sensed physiological sensing signal is not within the safe range. After the application is made (an adequate amount is usually added in advance), the procedure backs to step S100.

Wherein, the preset functional response enables the physiological sensing signal of the wearer returning to a stable state. If an initial application is insufficient, the wearable device keeps performing the physiological nursing, namely, a second application is made. The number of the nursing may be set in advance to enable the control device 110 executing the nursing procedures according to the practical situation or different physiological conditions.

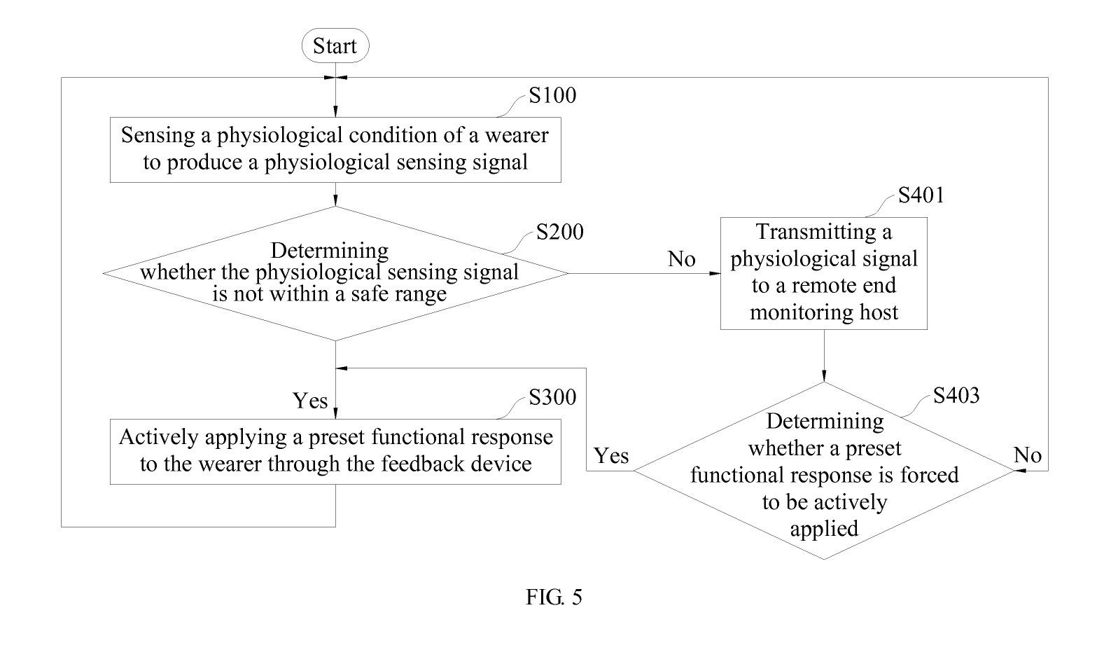

Please refer to FIG. 5 which is a flow chart of a physiological nursing method having active feedback characteristics in accordance with another embodiment of the present invention. The difference between FIG. 5 and FIG. 4 lies in that whether the physiological sensing signal is determined within a safe range is made in step S200. If the result is “positive”, step S 300 is entered, and if the result is “negative”, the procedure enters step S401. In step S401, a physiological signal is transmitted to a remote end monitoring host, wherein the remote end monitoring host is a monitoring host (i.e. the aforementioned second electronic communication device 300), and the monitoring host is stored with preset physiological data. Afterwards, step S403 is entered. The monitoring host determines whether the preset functional response has to be applied to the wearer corresponding to the preset physiological data and the physiological sensing signal when the physiological sensing signal is within the safe range. If the result is “positive”, the step S300 is entered, and if the result is “negative”, it goes to step S100. The extra step is determined by an added remote end monitoring host. For example, as the monitoring host is stored with a huge amount of data concerning identical physiological conditions, a more careful judgment mechanism is therefore provided. That to say, the monitoring host is stored with the historical data of the wearer, and the preset functional response relative to the wearer is hence forced to be actively applied through the feedback device in a certain particular cycle.

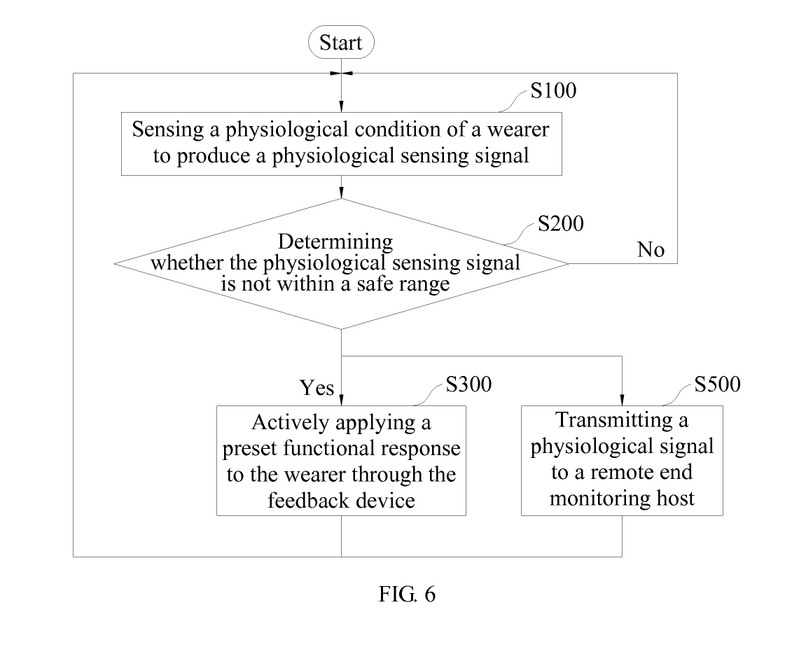

Please refer to FIG. 6 which is a flow chart of a physiological nursing method having active feedback characteristics in accordance with another embodiment of the present invention. The difference between FIG. 6 and FIG. 4 lies in that whether the physiological sensing signal is determined within a safe range is made in step S200. If the result is “positive”, it goes to step S300 and S500, and if the result is “negative”, it enters step S 100. Wherein, the physiological sensing signal is transmitted to the remote end in step S500. For example, the above-mentioned first electronic device is disposed with a global positioning system and a mobile communication module, and when the physiological sensing signal is not within the safe range, the first electronic communication device transmits the physiological sensing signal and position data produced by the global positioning system to the second electronic communication device (i.e. the remote end) through the mobile communication module. Wherein, the remote end is at least one of an electric communication device of a preset contactor and a nearby medical institute provided by the position data. As a result, when the physiological sensing signal is abnormal, a wearable device having active feedback characteristics for physiological nursing and a method having active feedback characteristics for physiological nursing in accordance with the present invention not only provide an adequate treatment in no time, but also inform the family or the nearby medicinal institute to obtain the follow-up aid.

While the means of specific embodiments in present invention has been described by reference drawings, numerous modifications and variations could be made thereto by those skilled in the art without departing from the scope and spirit of the invention set forth in the claims. The modifications and variations should in a range limited by the specification of the present invention.

What is claimed is:

1. A wearable device having active feedback characteristics for physiological nursing, comprising:
a physiological signal sensing device being wearable to a wearer, and a control device connected with the physiological signal sensing device; the wearable device having active feedback characteristics for physiological nursing further comprising a feedback device being wearable to the wearer, and the feedback device connected with the control device, the control device being configured to command the feedback device applying a preset functional response to the wearer when the control device determines a physiological sensing signal produced by the physiological signal sensing device that is not within a safe range, wherein the preset functional response enables the physiological sensing signal of the wearer returning to a stable state so as to be maintained within the safe range.

2. The wearable device of claim 1, wherein the feedback device comprises at least one of a sound player, a vibrator, a current releaser, and a medicine releaser, wherein a preset functional response of the sound player is to play a preset sound, a preset functional response of the vibrator is to produce a vibration, a preset functional response of the current releaser is to produce an effect of electric shock, and a preset functional response of the medicine releaser is to apply a medicine to the wearer.

3. The wearable device of claim 2, wherein the current releaser comprises an anode patch and a cathode patch adhered to the wearer, and the current releaser releases a low frequency current through the anode patch and the cathode patch.

4. The wearable device of claim 3, further comprising: a wireless communication module connected with the control device, wherein the wireless communication module is configured to connect with a first electronic communication device outside the wearable device having active feedback characteristics for physiological nursing such that the physiological signal is transmitted to the first electronic communication device.

5. The wearable device of claim 4, wherein the wireless communication module is a near field communication module or a far field communication module.

6. A physiological nursing system of claim 1, comprising:
a wearable device having active feedback characteristics for physiological nursing being wearable to a wearer, and configured to sense a physiological sensing signal of the wearer and actively commanding a feedback device to produce a preset functional response to the wearer when the physiological sensing signal is not within a safe range, and
a first electronic communication device connected with the wearable device having active feedback characteristics for physiological nursing to receive the physiological sensing signal and transmit the physiological sensing signal to a second electronic communication device located in a remote end.

7. The physiological nursing system of claim 6, wherein the first electronic communication device is disposed with a global positioning system and a mobile communication module, and when the physiological sensing signal is not within the safe range, the first electronic communication device transmits the physiological sensing signal and position data produced by the global positioning system to the second electronic communication device through the mobile communication module.

8. A physiological nursing system of claim 2, comprising:
a wearable device having active feedback characteristics for physiological nursing being wearable to a wearer, and configured to sense a physiological sensing signal of the wearer and actively commanding a feedback device to produce a preset functional response to the wearer when the physiological sensing signal is not within a safe range, and
a first electronic communication device connected with the wearable device having active feedback characteristics for physiological nursing to receive the physiological sensing signal and transmit the physiological sensing signal to a second electronic communication device located in a remote end.

9. The physiological nursing system of claim 8, wherein the first electronic communication device is disposed with a global positioning system and a mobile communication module, and when the physiological sensing signal is not within the safe range, the first electronic communication device transmits the physiological sensing signal and position data produced by the global positioning system to the second electronic communication device through the mobile communication module.

10. A physiological nursing system of claim 3, comprising:
a wearable device having active feedback characteristics for physiological nursing being wearable to a wearer, and configured to sense a physiological sensing signal of the wearer and actively commanding a feedback device to produce a preset functional response to the wearer when the physiological sensing signal is not within a safe range, and
a first electronic communication device connected with the wearable device having active feedback characteristics for physiological nursing to receive the physiological sensing signal and transmit the physiological sensing signal to a second electronic communication device located in a remote end.

11. The physiological nursing system of claim 10, wherein the first electronic communication device is disposed with a global positioning system and a mobile communication module, and when the physiological sensing signal is not within the safe range, the first electronic communication device transmits the physiological sensing signal and position data produced by the global positioning system to the second electronic communication device through the mobile communication module.

12. A physiological nursing system of claim 4, comprising:
a wearable device having active feedback characteristics for physiological nursing being wearable to a wearer, and configured to sense a physiological sensing signal of the wearer and actively commanding a feedback device to produce a preset functional response to the wearer when the physiological sensing signal is not within a safe range, and
a first electronic communication device connected with the wearable device having active feedback characteristics for physiological nursing to receive the physiological sensing signal and transmit the physiological sensing signal to a second electronic communication device located in a remote end.

13. The physiological nursing system of claim 12, wherein the first electronic communication device is disposed with a global positioning system and a mobile communication module, and when the physiological sensing signal is not within the safe range, the first electronic communication device transmits the physiological sensing signal and position data produced by the global positioning system to the second electronic communication device through the mobile communication module.

14. A physiological nursing system of claim 5, comprising:
a wearable device having active feedback characteristics for physiological nursing being wearable to a wearer, and configured to sense a physiological sensing signal of the wearer and actively commanding a feedback device to produce a preset functional response to the wearer when the physiological sensing signal is not within a safe range, and
a first electronic communication device connected with the wearable device having active feedback characteristics for physiological nursing to receive the physiological sensing signal and transmit the physiological sensing signal to a second electronic communication device located in a remote end.

15. The physiological nursing system of claim 14, wherein the first electronic communication device is disposed with a global positioning system and a mobile communication module, and when the physiological sensing signal is not within the safe range, the first electronic communication device transmits the physiological sensing signal and position data produced by the global positioning system to the second electronic communication device through the mobile communication module.

16. A method having active feedback characteristics for physiological nursing, comprising:
sensing a physiological condition of a wearer to produce a physiological sensing signal,
determining whether the physiological sensing signal is within a safe range, and
actively applying a preset functional response relative to the physiological condition of the wearer through a feedback device when the physiological sensing signal is not within the safe range, wherein the preset functional response enables the physiological sensing signal of the wearer returning to a stable state.

17. The method of claim 16, wherein the preset functional response comprises at least one of playing a preset sound, producing a vibration, producing a low frequency electronic shock, and applying a medicine to the wearer.

18. The method of claim 17, further comprising: producing position data through a global positioning system, and simultaneously transmitting the physiological sensing signal to a remote end as an alert when the physiological sensing signal is not within the safe range, wherein the remote end is at least one of an electric communication device of a preset contactor and a nearby medical institute provided by the position data.

19. The method of claim 17, in addition to actively applying a preset functional response relative to the physiological condition of the wearer when the physiological sensing signal is not within the safe range, the method further comprising: regularly transmitting the physiological sensing signal to the remote end, wherein the remote end is a monitoring host stored with preset physiological data, and the monitoring host determines whether the preset functional response has to be applied to the wearer corresponding to the preset physiological data and the physiological sensing signal when the physiological sensing signal is within the safe range; and if a result is positive, the preset functional response relative to the wearer is forced to be actively applied through the feedback device.
